(19) United States
(12) Patent Application Publication
(10) Pub. No.: US 2026/0041732 A1
(43) Pub. Date: Feb. 12, 2026

(54) USE OF APELIN FOR THE TREATMENT OF LYMPHEDEMA

(71) Applicants: INSTITUT NATIONAL DE LA SANTE ET DE LA RECHERCHE MEDICALE, Paris (FR); CENTRE HOSPITALIER UNIVERSITAIRE DE TOULOUSE, Toulouse (FR); UNIVERSITE TOULOUSE III – PAUL SABATIER, Toulouse (FR); FLASH THERAPEUTICS, Toulouse (FR)

(72) Inventors: Barbara GARMY-SUSINI, Toulouse (FR); Alessandra BURA RIVIERE, Toulouse (FR); Julie MALLOIZEL, Toulouse (FR); Anne-Catherine PRATS, Toulouse (FR)

(21) Appl. No.: 18/998,395

(22) PCT Filed: Jul. 24, 2023
(86) PCT No.: PCT/EP2023/070478

§ 371 Date: Jan. 24, 2025

(30) Foreign Application Priority Data
Jul. 25, 2022 (EP) 22306106.0

(51) Int. Cl. A61K 38/17; A61K 35/76; A61K 38/18; A61K 48/00; A61P 7/10; C12N 15/86

(52) U.S. Cl. CPC A61K 38/1796; A61K 35/76; A61K 38/1866; A61K 48/005; A61P 7/10; C12N 15/86

(57) ABSTRACT

Lymphedema is a chronic condition causes by a lymphatic dysfunction that leads to the accumulation of fluid and fat in the limb. Here, the inventors identified the bioactive peptide apelin as good candidate to restore the lymphatic flow in lymphedema. They found a significant decrease in apelin expression in women lymphedematous arm compared to their normal arm. Using an apelin-knockout mouse model, they confirmed the crucial role of apelin as lymphedema was maintained more than 4 weeks after surgery and was associated with a lack of dermis lymphangiogenesis and increased dermal backflow. The inventors show that intradermal injection of apelin-lentivector significantly reduced limb swelling. This was associated with a reduction of dermis fibrosis and increase in lymphatic density. Importantly, apelin stimulates eNOS-mediated lymphatic pumping via Akt and eNOS phosphorylation in lymphatic endothelial cells (LEC). This was associated with significant increase in E2F8-targeted gene expression through the direct binding of E2F8 on CCBE1 promoter in LEC. Taken together, the results show that apelin plays a key role in lymphedema and thus represents a novel partner for VEGF-C to prevent limb swelling and tissue fibrosis in lymphedema.

### FIELD OF THE INVENTION

The present invention is in the field of medicine, in particular treatment of lymphedema.

### BACKGROUND OF THE INVENTION

Lymphedema is a multifactorial condition that substantially affects the quality of life of patients (Greene, Grant et al. 2012, Hoffner, Peterson et al. 2018). It is characterized by a failure of the lymph transport back to the blood circulation due to a lymphatic dysfunction that occurs after genetic mutation (primary lymphedema) or arises after cancer treatments (surgery, radiations, chemotherapies . . . ) (Mortimer and Rockson 2014).

Lymph stasis promotes changes in surrounding tissues leading to adipose tissue accumulation and severe fibrosis. The adipose tissue deposition has been attributed to a stimulation of adipogenesis in the affected limb (Mortimer and Rockson 2014). Recent evidences highlighted a novel concept showing an imbalance between adipose tissue de novo genesis and lipolysis suggesting major changes in adipokine synthesis (Koc, Wald et al. 2021, Sano, Hirakawa et al. 2022). Also, a major hallmark of lymphedema associated with lymph stasis is the development of fibrosis in the skin and in the adipose tissue (Mortimer and Rockson 2014). Lymphostatic fibrosis defines the stage of lymphedema from reversible to elephantiasis stages. To date, there is no cure for lymphedema except massages and compressive bandagings. However, once fibrosis develops, tissues become denser leading to a lymphatic vessels obstruction that worsen lymphedema. Importantly, fibrosis also affects collecting lymphatic pumping and increases limb swelling (Baik, Park et al. 2022) (Kataru, Wiser et al. 2019).

A large number of cytokines and peptides are selectively involved adipocyte metabolism, endothelial function or tissue fibrosis. However, the bioactive peptide apelin combines the beneficial effects on the limb tissues as a whole. Apelin is the endogenous ligand of the G-protein-coupled receptor APJ that is expressed in a diverse number of organs (Pope, Roberts et al. 2012). The first evidences of the link between apelin and lymphatic vasculature were identified in a tumoral environment as apelin stimulates both hemangio- and lymphangiogenesis (Berta, Hoda et al. 2014). Also, our group previously described the beneficial effect of apelin on cardiac lymphatic vasculature after heart ischemia (Tatin, Renaud-Gabardos et al. 2017). We found that apelin was able to restore the lymphatic shape of precollecting vessels in the infarct area suggesting that apelin may represent a good candidate to restore the lymphatic shape in injured tissues (Tatin, Renaud-Gabardos et al. 2017). However, apelin was first described as an adipokine synthesized by white adipose tissue and other organs such as heart, kidney and central nervous system (Castan-Laurell, Boucher et al. 2005) (Dai, Smith et al. 2013). The production and secretion of apelin by adipocytes is controlled by insulin and its beneficial role on the adipose tissue has emerged as a promising target in obesity and diabetes (Castan-Laurell, Boucher et al. 2005). Apelin is also a critical actor of the fibrosis protection in many organs including heart, lung and kidney (Huang, Chen et al. 2016). Apelin inhibits the occurrence of myocardial fibrosis after ischemia and atrial fibrillation by blocking the Ang2 pathway. In the heart, it participates to maintain proper lymphatic vessel shape after ischemia (Tatin, Renaud-Gabardos et al. 2017). It decreases renal and skin fibrosis by inhibiting the TGFβ signaling Yokoyama, Sekiguchi et al. 2018). Surprisingly, the protective effect of apelin on the white adipose tissue fibrosis has been poorly investigated. In particular, the beneficial effect of apelin on diet-induced obesity has been attributed to its ability to improve the lymphatic and blood vessel integrity (Sawane, Kajiya et al. 2013). Apelin stimulates NO production via PI3K/Akt signalling in blood endothelial cells (Busch, Strohbach et al. 2015).

### SUMMARY OF THE INVENTION

The present invention is defined by the claims. In particular, the present invention relates to the use of apelin for the treatment of lymphedema.

### DETAILED DESCRIPTION OF THE INVENTION

Lymphedema is a chronic condition causes by a lymphatic dysfunction that leads to the accumulation of fluid and fat in the limb. It is a characterized by a severe inflammation and fibrosis affecting the ability to move and increasing the risks of skin infections. Here, the inventors identified the bioactive peptide apelin as good candidate to restore the lymphatic flow in lymphedema. They found a significant decrease in apelin expression in women lymphedematous arm compared to their normal arm. Using an apelin-knockout mouse model, they confirmed the crucial role of apelin as lymphedema was maintained more than 4 weeks after surgery and was associated with a lack of dermis lymphangiogenesis and increased dermal backflow. The inventors show that intradermal injection of apelin-lentivector significantly reduced limb swelling. This was associated with a reduction of dermis fibrosis and increase in lymphatic density. Importantly, apelin stimulates eNOS-mediated lymphatic pumping via Akt and eNOS phosphorylation in lymphatic endothelial cells (LEC). This was associated with significant increase in E2F8-targeted gene expression through the direct binding of E2F8 on CCBE1 promoter in LEC. Taken together, the results show that apelin plays a key role in lymphedema and thus represents a novel partner for VEGF-C to prevent limb swelling and tissue fibrosis in lymphedema.

Accordingly, the first object of the present invention relates to a method of treating lymphedema in a patient in need thereof comprising administering to the patient a therapeutically effective amount of i) an apelin polypeptide or ii) a polynucleotide encoding an apelin polypeptide.

As used herein, the term “lymphedema” has its general meaning in the art and refers to a disorder characterized by a strong tissue swelling due to an increased fluid retention in the tissue, a local accumulation of adipose tissue and an impairment of immune function due to reduced lymphatic drainage. The term includes “primary lymphedema” and “secondary lymphedema”. Primary lymphedema is a lymphatic system malformation characterized by swelling of an extremity that can be associated with other lymphatic effusions, due to an underlying developmental anomaly of the lymphatic system (abnormal lymphangiogenesis). It can be hereditary or not and be congenital or late onset. In some embodiments, lymphedema is found as secondary disorder which may result from lymph node dissection or injury of lymphatic vessels. Such secondary lymphedema may also accompany, e.g., lymph node dissection and injury in connection with surgery, radiation therapy, tumor disease and treatments thereof, musculoskeletal injuries like fractures, tendon releases and joint replacements, neurological conditions like muscle paresis, vascular injuries/surgeries, integumentary injuries, coagulation disorders such as deep vein thrombosis, scar tissue formation, tamoxifen treatment, filariasis, infection, lipedema or cellulitis.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

In particular, the method of the present invention is particularly suitable for increasing lymphatic vessel plasticity, contractility and/or dilatation.

As used herein, the term “apelin” has its general meaning in the art and indicates a 77 residue preprotein (NCBI Reference Sequence: NP—0059109.3, and encoded by NCBI Reference Sequence: NM—017413.3), which gets processed into biologically active forms of apelin peptides, such as apelin-36, apelin-17, apelin-16, apelin-13, apelin-12. The full length mature peptide, referred to as “apelin-36,” comprises 36 amino acids, but the most potent isoform is the pyroglutamated form of a 13mer of apelin (apelin-13), referred to as “Pyr-1-apelin-13 or Pyr1-apelin-13” Different apelin forms are described, for instance, in U.S. Pat. No. 6,492,324B1. An exemplary amino acid sequence for Apelin is represented by SEQ ID NO:1.

SEQ ID NO: 1> sp|Q9ULZ1|APEL_HUMAN Apelin OS =
Homo sapiens O X = 9606 GN = APLN PE = 2 SV = 1
MNLRLCVQALLLLWLSLTAVCGGSLMPLPDGNGLEDGNVRHLVQPRGSR
NGPGPWQGGRRKFRRQRPRLSHKGPMPF

As used herein, the term “polypeptide” has its general meaning in the art and refers to a polymer of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art.

As used herein, the term “apelin polypeptide” refers to a polypeptide that comprises an amino acid sequence having at least 90% of identity with the amino acid sequence as set forth in SEQ ID NO: 1.

As used herein, the “percent identity” between two sequences is a function of the number of identical positions shared by the sequences (i.e., % identity=number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described below. The percent identity between two amino acid sequences can be determined using the Needleman and Wunsch algorithm (Needleman, Saul B. & Wunsch, Christian D. (1970). “A general method applicable to the search for similarities in the amino acid sequence of two proteins”. Journal of Molecular Biology. 48 (3): 443-53.). The percent identity between two nucleotide or amino acid sequences may also be determined using for example algorithms such as EMBOSS Needle (pair wise alignment; available at www.ebi.ac.uk). For example, EMBOSS Needle may be used with a BLOSUM62 matrix, a “gap open penalty” of 10, a “gap extend penalty” of 0.5, a false “end gap penalty”, an “end gap open penalty” of 10 and an “end gap extend penalty” of 0.5. In general, the “percent identity” is a function of the number of matching positions divided by the number of positions compared and multiplied by 100. For instance, if 6 out of 10 sequence positions are identical between the two compared sequences after alignment, then the identity is 60%. The % identity is typically determined over the whole length of the query sequence on which the analysis is performed. Two molecules having the same primary amino acid sequence or nucleic acid sequence are identical irrespective of any chemical and/or biological modification. According to the invention a first amino acid sequence having at least 90% of identity with a second amino acid sequence means that the first sequence has 90; 91; 92; 93; 94; 95; 96; 97; 98; 99 or 100% of identity with the second amino acid sequence.

As used herein, the term “polynucleotide” refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the term “polynucleotide” includes polydeoxyribonucleotides (containing 2-deoxy-D-ribose), polyribonucleotides (containing D-ribose), including tRNA, rRNA, hRNA, siRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA. In some embodiments, the polynucleotide comprises an mRNA. In other aspect, the mRNA is a synthetic mRNA. In some embodiments, the synthetic mRNA comprises at least one unnatural nucleobase. In some embodiments, all nucleobases of a certain class have been replaced with unnatural nucleobases (e.g., all uridines in a polynucleotide disclosed herein can be replaced with an unnatural nucleobase, e.g., 5-methoxyuridine). In some embodiments, the polynucleotide (e.g., a synthetic RNA or a synthetic DNA) comprises only natural nucleobases, i.c., A, C, T and G in the case of a synthetic DNA, or A, C, T, and U in the case of a synthetic RNA.

In some embodiments, the polynucleotide of the present invention is a messenger RNA (mRNA).

In some embodiments, the polynucleotide is inserted in a vector, such a viral vector.

As used herein, the term “viral vector” refers to a virion or virus particle that functions as a nucleic acid delivery vehicle and which comprises a vector genome packaged within the virion or virus particle. Typically, the vector is a viral vector which is an adeno-associated virus (AAV), a retroviral vector, bovine papilloma virus, an adenovirus vector, a vaccinia virus, or a polyoma virus.

In some embodiments, the viral vector is a AAV vector.

As used herein, the term “AAV vector” means a vector derived from an adeno-associated virus serotype, including without limitation, AAV1, AAV2, AAV3, AAV4, AAV5, AAV6, AAV7, AAV8, AAV9, and mutated forms thereof. AAV vectors can have one or more of the AAV wild-type genes deleted in whole or part, preferably the rep and/or cap genes, but retain functional flanking ITR sequences.

In some embodiments, the viral vector is a retroviral vector.

As used herein, the term “retroviral vector” refers to a vector containing structural and functional genetic elements that are primarily derived from a retrovirus.

In some embodiments, the retroviral vector of the present invention derives from a retrovirus selected from the group consisting of alpharetroviruses (e.g., avian leukosis virus), betaretroviruses (e.g., mouse mammary tumor virus), gammaretroviruses (e.g., murine leukemia virus), deltaretroviruses (e.g., bovine leukemia virus), epsilonretroviruses (e.g., Walley dermal sarcoma virus), lentiviruses (e.g., HIV-1, HIV-2) and spumaviruses (e.g., human spumavirus).

In some embodiments, the retroviral vector of the present invention is a replication deficient retroviral virus particle, which can transfer a foreign imported RNA of a gene instead of the retroviral mRNA.

In some embodiments, the retroviral vector of the present invention is a lentiviral vector.

As used herein, the term “lentiviral vector” refers to a vector containing structural and functional genetic elements that are primarily derived from a lentivirus. In some embodiments, the lentiviral vector of the present invention is selected from the group consisting of HIV-1, HIV-2, SIV, FIV, EIAV, BIV, VISNA and CAEV vectors. In some embodiments, the lentiviral vector is a HIV-1 vector.

The structure and composition of the vector genome used to prepare the retroviral vectors of the present invention are in accordance with those described in the art. Especially, minimum retroviral gene delivery vectors can be prepared from a vector genome, which only contains, apart from the recombinant nucleic acid molecule of the present invention, the sequences of the retroviral genome which are non-coding regions of said genome, necessary to provide recognition signals for DNA or RNA synthesis and processing. In some embodiment, the retroviral vector genome comprises all the elements necessary for the nucleic import and the correct expression of the polynucleotide of interest (i.e. the transgene). As examples of elements that can be inserted in the retroviral genome of the retroviral vector of the present invention are at least one (preferably two) long terminal repeats (LTR), such as a LTR5′ and a LTR3′, a psi sequence involved in the retroviral genome encapsidation, and optionally at least one DNA flap comprising a cPPT and a CTS domains. In some embodiments of the present invention, the LTR, preferably the LTR3′, is deleted for the promoter and the enhancer of U3 and is replaced by a minimal promoter allowing transcription during vector production while an internal promoter is added to allow expression of the transgene. In particular, the vector is a Self-INactivating (SIN) vector that contains a non-functional or modified 3′ Long Terminal Repeat (LTR) sequence. This sequence is copied to the 5′ end of the vector genome during integration, resulting in the inactivation of promoter activity by both LTRs. Hence, a vector genome may be a replacement vector in which all the viral coding sequences between the 2 long terminal repeats (LTRs) have been replaced by the recombinant nucleic acid molecule of the present invention.

In some embodiments, the retroviral vector genome is devoid of functional gag, pol and/or env retroviral genes. By “functional” it is meant a gene that is correctly transcribed, and/or correctly expressed. Thus, the retroviral vector genome of the present invention in this embodiment contains at least one of the gag, pol and env genes that is either not transcribed or incompletely transcribed; the expression “incompletely transcribed” refers to the alteration in the transcripts gag, gag-pro or gag-pro-pol, one of these or several of these being not transcribed. In some embodiments, the retroviral genome is devoid of gag, pol and/or env retroviral genes.

In some embodiments the retroviral vector genome is also devoid of the coding sequences for Vif-, Vpr-, Vpu-and Nef-accessory genes (for HIV-1 retroviral vectors), or of their complete or functional genes.

Typically, the retroviral vector of the present invention is non replicative i.e., the vector and retroviral vector genome are not able to form new particles budding from the infected host cell. This may be achieved by the absence in the retroviral genome of the gag, pol or env genes, as indicated in the above paragraph; this can also be achieved by deleting other viral coding sequence(s) and/or cis-acting genetic elements needed for particles formation.

Thus the present invention encompasses use of virus-like particles. As used herein, the term “virus-like particle” or “VLP” refers to a structure resembling a virus particle but devoid of the viral genome, incapable of replication and devoid of pathogenicity. The particle typically comprises at least one type of structural protein from a virus. Preferably only one type of structural protein is present. Most preferably no other non-structural component of a virus is present. Thus, virus-like particles can be spontaneously self-assembled by viral structural proteins under appropriate conditions in vitro while excluding the genetic material and potential replication probability. virus-like particles, with a diameter of approximately 20 to 150 nm, also have the characteristics of nanometer materials, such as large surface area, surface-accessible amino acids with reactive moieties (e.g., lysine and glutamic acid residues), inerratic spatial structure, and good biocompatibility. Therefore, assembled virus-like particles have great potential as a delivery system for specifically carrying a variety of cargos. In some embodiments, one or more of the zinc finger motifs of the Gag protein is/are substituted by one or more RNA-binding domain(s). In some embodiments, the RNA-binding domain is the Coat protein of the MS2 bacteriophage, of the PP7 phage or of the Q3 phage, the prophage HK022 Nun protein, the ULA protein or the hPum protein. More preferably, the RNA binding domain is the Coat protein of the MS2 bacteriophage or of the PP7 phage. Even more preferably the RNA-binding domain is the Coat protein of the MS2 bacteriophage. These embodiments are particularly suitable for packaging the mRNA encoding for the apelin polypeptide into the VLP. Thus, in some embodiments, the mRNA encoding for the apelin polypeptide that is encapsuled in the virus particle of the present invention comprises at least one encapsidation sequence. By “encapsidation sequence” is meant an RNA motif (sequence and three-dimensional structure) recognized specifically by an RNA-binding domain as above described. Preferably, the encapsidation sequence is a stem-loop motif. Even more preferably, the encapsidation sequence of the retroviral particle is the stem-loop motif of the RNA of the MS2 bacteriophage or of the PP7 phage such as. The stem-loop motif and more particularly the stem-loop motif of the RNA of the MS2 bacteriophage or that of the RNA of the PP7 phage may be used alone or repeated several times, preferably from 2 to 25 times, more preferably from 2 to 18 times, for example from 6 to 18 times. In some embodiments, the present invention encompasses the use of the LentiFlash® technology that based on non-integrative lentiviral particles constructed using a bacteriophage coat protein and its cognate 19-nt stem loop, to replace the natural lentiviral Psi packaging sequence, in order to achieve active mRNA packaging into the lentiviral particles (Prel A, Caval V, Gayon R, Ravassard P, Duthoit C, Payen E, Maouche-Chretien L, Creneguy A, Nguyen T H, Martin N, Piver E, Sevrain R, Lamouroux L, Leboulch P, Deschaseaux F, Bouillé P, Sensébé L, Pagès J C. Highly efficient in vitro and in vivo delivery of functional RNAs using new versatile MS2-chimeric retrovirus-like particles. Mol Ther Methods Clin Dev. 2015 Oct. 21; 2:15039. doi: 10.1038/mtm.2015.39. PMID: 26528487; PMCID: PMC4613645).

The retroviral vectors of the present invention can be produced by any well-known method in the art including by transfection(s) transient(s), in stable cell lines and/or by means of helper virus. Use of stable cell lines may also be preferred for the production of the vectors (Greene, M. R. et al. Transduction of Human CD34+Repopulating Cells with a Self-Inactivating Lentiviral Vector for SCID-X1 Produced at Clinical Scale by a Stable Cell Line. Hum. Gene Ther. Methods 23, 297-308 (2012).) For instance, the retroviral vector of the present invention is obtainable by a transcomplementation system (vector/packaging system) by transfecting in vitro a permissive cell (such as 293T cells) with a plasmid containing the retroviral vector genome of the present invention, and at least one other plasmid providing, in trans, the gag, pol and env sequences encoding the polypeptides GAG, POL and the envelope protein(s), or for a portion of these polypeptides sufficient to enable formation of retroviral particles. As an example, permissive cells are transfected with a) transcomplementation plasmid, lacking packaging signal psi and, the plasmid is optionally deleted of accessory genes vif, nef, vpu and/or vpr, b) a second plasmid (envelope expression plasmid or pseudotyping env plasmid) comprising a gene encoding an envelope protein(s) and c) a plasmid vector comprising a recombinant genome retroviral, optionally deleted from the promoter region of the 3′ LTR or U3 enhancer sequence of the 3′ LTR, including, between the LTR sequences 5′ and 3′ retroviral, a psi encapsidation sequence, a nuclear export element (preferably RRE element of HIV or other retroviruses equivalent), comprising the nucleic acid molecule of the present invention and optionally a promoter and/or a nuclear import sequence (cPPT sequence eg CTS) of the RNA. Advantageously, the three plasmids used do not contain homologous sequence sufficient for recombination. Nucleic acids encoding gag, pol and env cDNA can be advantageously prepared according to conventional techniques, from viral gene sequences available in the prior art and databases. The trans-complementation plasmid provides a nucleic acid encoding the proteins retroviral gag and pol. These proteins are derived from a lentivirus, and most preferably, from HIV-1. The plasmid is devoid of encapsidation sequence, sequence coding for an envelope, accessory genes, and advantageously also lacks retroviral LTRs. Therefore, the sequences coding for gag and pol proteins are advantageously placed under the control of a heterologous promoter, eg cellular, viral, etc . . . , which can be constitutive or regulated, weak or strong. It is preferably a plasmid containing a sequence transcomplémentant Δpsi-CMV-gag-pol-PolyA. This plasmid allows the expression of all the proteins necessary for the formation of empty virions, except the envelope glycoproteins. The plasmid transcomplementation may advantageously comprise the TAT and REV genes. Plasmid transcomplementation is advantageously devoid of vif, vpr, vpu and/or nef accessory genes. It is understood that the gag and pol genes and genes TAT and REV can also be carried by different plasmids, possibly separated. In this case, several plasmids are used transcomplementation, each encoding one or more of said proteins. The promoters used in the plasmid transcomplementation, the envelope plasmid and the plasmid vector respectively to promote the expression of gag and pol of the coat protein, the mRNA of the vector genome and the transgene are promoters identical or different, chosen advantageously from ubiquitous promoters or specific, for example, from viral promoters CMV, TK, RSV LTR promoter and the RNA polymerase III promoter such as U6 or H1 or promoters of helper viruses encoding env, gag and pol (i.e. adenoviral, baculoviral, herpes viruses). For the production of the retroviral vector of the present invention, the plasmids described above can be introduced into competent cells and viruses produced are harvested. The cells used may be any cell competent, particularly eukaryotic cells, in particular mammalian, eg human or animal. They can be somatic or embryonic stem or differentiated. Typically the cells include 293T cells, fibroblast cells, hepatocytes, muscle cells (skeletal, cardiac, smooth, blood vessel, etc.)., nerve cells (neurons, glial cells, astrocytes) of epithelial cells, renal, ocular etc . . . It may also include, insect, plant cells, yeast, or prokaryotic cells. It can also be cells transformed by the SV40 T antigen. The genes gag, pol and env encoded in plasmids or helper viruses can be introduced into cells by any method known in the art, suitable for cell type considered. Usually, the cells and the vector system are contacted in a suitable device (plate, dish, tube, pouch, etc . . . ), for a period of time sufficient to allow the transfer of the vector system or the plasmid in the cells. Typically, the vector system or the plasmid is introduced into the cells by calcium phosphate precipitation, electroporation, transduction or by using one of transfection-facilitating compounds, such as lipids, polymers, liposomes and peptides, etc. The calcium phosphate precipitation is preferred. The cells are cultured in any suitable medium such as RPMI, DMEM, a specific medium to a culture in the absence of fetal calf serum, etc. Once transfected the retroviral vectors of the present invention may be purified from the supernatant of the cells. Purification of the retroviral vector to enhance the concentration can be accomplished by any suitable method, such as by density gradient purification (e.g., cesium chloride (CsCl)) or by chromatography techniques (e.g., column or batch chromatography). For example, the vector of the present invention can be subjected to two or three CsCl density gradient purification steps. The vector, is desirably purified from cells infected using a method that comprises lysing cells infected with adenovirus, applying the lysate to a chromatography resin, eluting the adenovirus from the chromatography resin, and collecting a fraction containing the retroviral vector of the present invention.

In some embodiments, the vector of the present invention includes “control sequences”, which refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell. Another nucleic acid sequence, is a “promoter” sequence, which is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters”.

In some embodiments, the polypeptide or polynucleotide of the present invention can be conjugated to at least one other molecule. Typically, said molecule is selected from the group consisting of polynucleotides, polypeptides, lipids, lectins, carbohydrates, vitamins, cofactors, and drugs. In some embodiments, the polypeptide or polynucleotide of the present invention is formulated using one or more lipid-based structures that include but are not limited to liposomes, lipoplexes, or lipid nanoparticles (Paunovska, Kalina, David Loughrey, and James E. Dahlman. “Drug delivery systems for RNA therapeutics.” Nature Reviews Genetics (2022): 1-16). Liposomes are artificially-prepared vesicles which can primarily be composed of a lipid bilayer and can be used as a delivery vehicle for the administration of pharmaceutical formulations. Liposomes can be of different sizes such as, but not limited to, a multilamellar vesicle (MLV) which can be hundreds of nanometers in diameter and can contain a series of concentric bilayers separated by narrow aqueous compartments, a small unicellular vesicle (SUV) which can be smaller than 50 nm in diameter, and a large unilamellar vesicle (LUV) which can be between 50 and 500 nm in diameter. Liposome design can include, but is not limited to, opsonins or ligands in order to improve the attachment of liposomes to unhealthy tissue or to activate events such as, but not limited to, endocytosis. Liposomes can contain a low or a high pH in order to improve the delivery of the pharmaceutical formulations. As a non-limiting example, liposomes such as synthetic membrane vesicles are prepared by the methods, apparatus and devices described in U.S. Patent Publication Nos. 20130177638, 20130177637, 20130177636, 20130177635, 20130177634, 20130177633, 20130183375, 20130183373 and 20130183372. In some embodiments, the liposomes are formed from 1,2-diolcyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinolcyl-4-(2-dimethylaminoethyl)-[1,3]-dioxolanc (DLin-KC2-DMA), and MC3 (as described in U.S.20100324120) and liposomes which can deliver small molecule drugs such as, but not limited to, DOXIL® from Janssen Biotech, Inc. (Horsham, Pa.). The polypeptide of polynucleotide of the present invention can be encapsulated by the liposome and/or it can be contained in an aqueous core which can then be encapsulated by the liposome (see International Pub. Nos. WO2012031046, WO2012031043, WO2012030901 and WO2012006378 and U.S. Patent Publication Nos. 20130189351, 20130195969 and 20130202684). In some embodiments, the polynucleotide of the present invention is formulated with stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999 6:271-281; Zhang et al. Gene Therapy. 1999 6:1438-1447; Jeffs et al. Pharm Res. 2005 22:362-372; Morrissey et al., Nat Biotechnol. 2005 2:1002-1007; Zimmermann et al., Nature. 2006 441:111-114; Heyes et al. J Contr Rel. 2005 107:276-287; Semple et al. Nature Biotech. 2010 28:172-176; Judge et al. J Clin Invest. 2009 119:661-673; deFougerolles Hum Gene Ther. 2008 19:125-132; U.S. Patent Publication No. 20130122104).

In some embodiments, the polypeptide or polynucleotide of the present invention is administered in combination or association with a VEGF-C polypeptide or ii) a polynucleotide encoding a VEGF-C polypeptide.

As used herein, the term “VEGF-C” has its general meaning in the art and refers to the vascular endothelial growth factor C encoded by the VEGF-C gene. An exemplary amino acid sequence for VEGF-C is represented by SEQ ID NO:2.

SEQ ID NO: 2 > sp|P49767|VEGFC_HUMAN Vascular
endothelial growth factor C OS = Homo sapiens
OX = 9606 GN = VEGFC PE = 1 SV = 1
MHLLGFFSVACSLLAAALLPGPREAPAAAAAFESGLDLSDAEPDAGEAT
AYASKDLEEQLRSVSSVDELMTVLYPEYWKMYKCQLRKGGWQHNREQAN
LNSRTEETIKFAAAHYNTEILKSIDNEWRKTQCMPREVCIDVGKEFGVA
TNTFFKPPCVSVYRCGGCCNSEGLQCMNTSTSYLSKTLFEITVPLSQGP
KPVTISFANHTSCRCMSKLDVYRQVHSIIRRSLPATLPQCQAANKTCPT
NYMWNNHICRCLAQEDFMFSSDAGDDSTDGFHDICGPNKELDEETCQCV
CRAGLRPASCGPHKELDRNSCQCVCKNKLFPSQCGANREFDENTCQCVC
KRTCPRNQPLNPGKCACECTESPQKCLLKGKKFHHQTCSCYRRPCTNRQ
KACEPGFSYSEEVCRCVPSYWKRPQMS

In some embodiments, the both polypeptides or polynucleotides may be administered to the patient separately or in the same composition. For instance, bicistronic polynucleotide that encodes both for the Apelin polypeptide and VEGF-C polypeptide can be used. In some embodiments, the polynucleotides encoding for the polypeptides can be inserted/included in the same vectors (e.g. viral vector, virus-like particles . . . ).

As used herein, the expression “therapeutically effective amount” means a sufficient amount of the active ingredient for treating or reducing the symptoms at reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination with the active ingredients; and like factors well known in the medical arts. For example, it is well within the skill of the art to start doses of the compound at levels lower than those required to achieve the desired therapeutic effect and to gradually increase the dosage until the desired effect is achieved.

Typically the active ingredient of the present invention (i.e. the polypeptide or polynucleotide) is combined with pharmaceutically acceptable excipients, and optionally sustained-release matrices, such as biodegradable polymers, to form pharmaceutical compositions. The term “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The invention will be further illustrated by the following figures and examples. However, these examples and figures should not be interpreted in any way as limiting the scope of the present invention.

### FIGURES

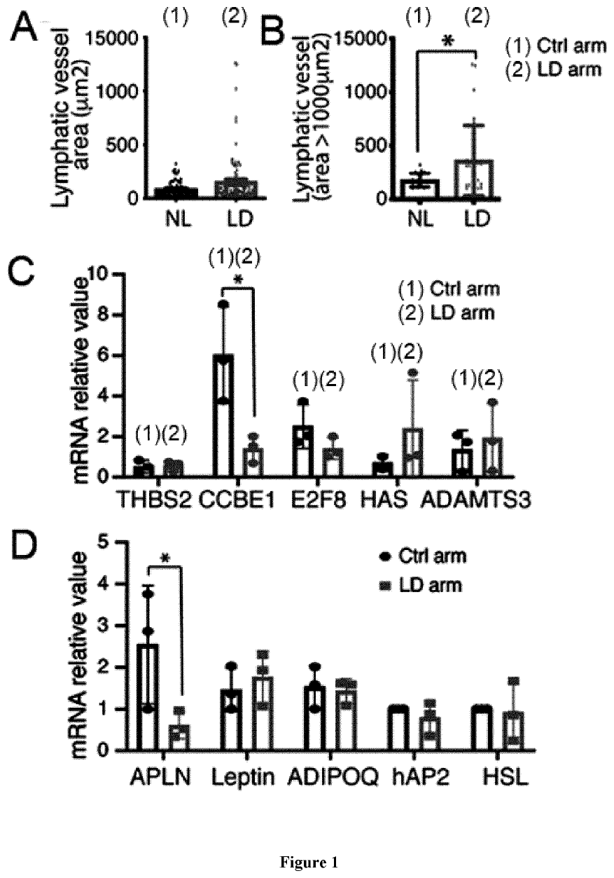

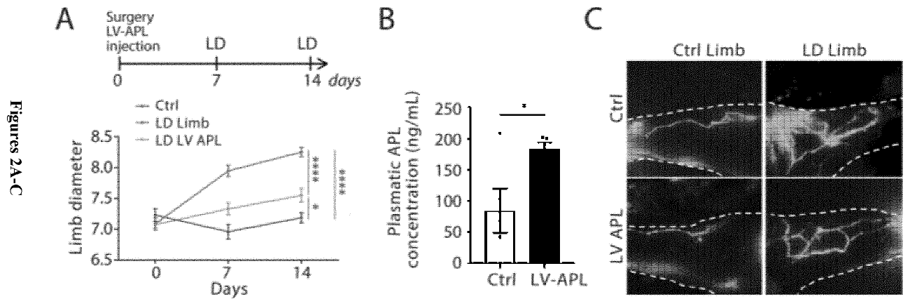

FIG. 1: Reduced Apelin expression in human lymphedema
A. Quantification of the lymphatic vessel diameters (*p<0.05). B. Quantification of the dermis lymphatic density (p<0.05). C. Quantitative RT-PCR analysis of the genes involved in fibrosis and VEGFC maturation in dermolipectomy samples from patients with lymphedema (*p<0.05)(n=3). D. Quantitative RT-PCR analysis of the adipokines in dermolipectomy samples from patients with lymphedema (*p<0.05)(n=3).

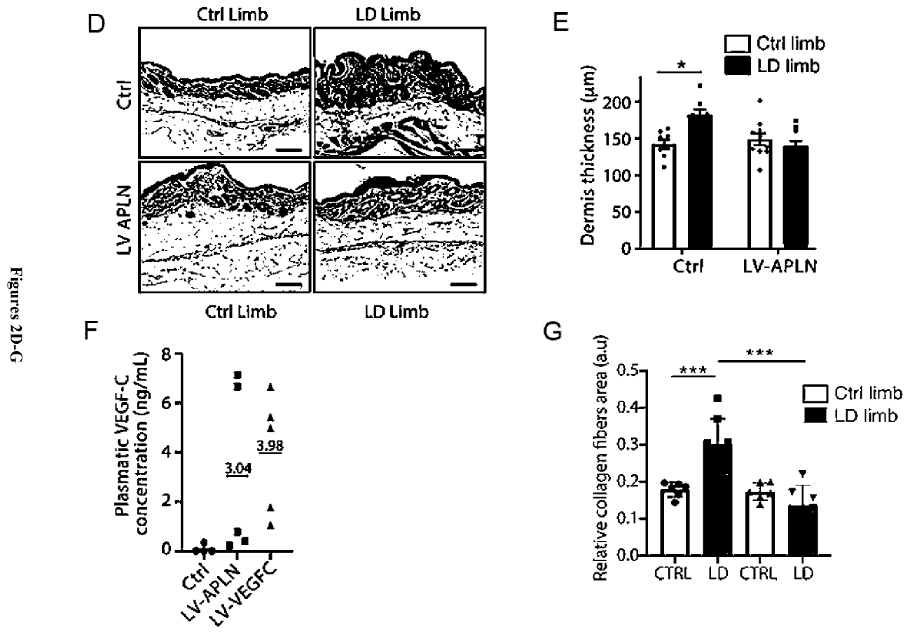

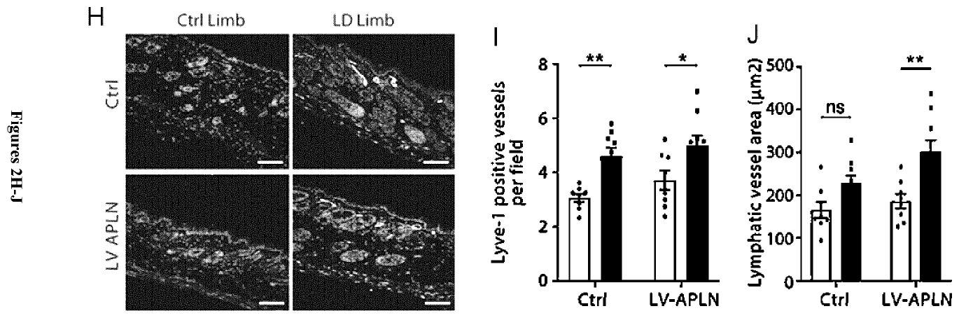

FIG. 2. Apelin prevents secondary lymphedema
A. Schematic of the experimental design of secondary lymphedema model in mice injected Apelin lentivector (LV-APL). Quantification of proximal leg swelling at 7 and 14 days after surgery on control limb, lymphedema limb (n=17) or lymphedema treated with apelin lentivector (n=20)(*p<0.05). B. EIA dosage of circulating apelin in plasma of control (n=5) or apelin treated mice (n=5). C. Lymphangiography reveals pathological remodeling of lymphatic vessels and dermal backflow in lymphedema that is reversed by LV-APL (n=10). D. Masson's trichrome staining of the skin from mice with lymphedema treated or not with Apelin (scale bar: 50 μm). E. Quantification of dermis thickness (*p<0.05). F. EIA dosage of circulating VEGF-C in plasma of control (n=5) or apelin-treated mice (n=5). G. SHG signal from deep, collagen-rich layer within dermis. H. Lyve-1 immunodetection of the skin lymphangiogenesis in apelin-treated mice (scale bar: 50 μm). I. Quantification of lymphangiogenesis in apelin-treated mice (*p<0.05, ** p<0.01). J. Quantification of lymphatic dilatation in apelin-treated mice (** p<0.01).

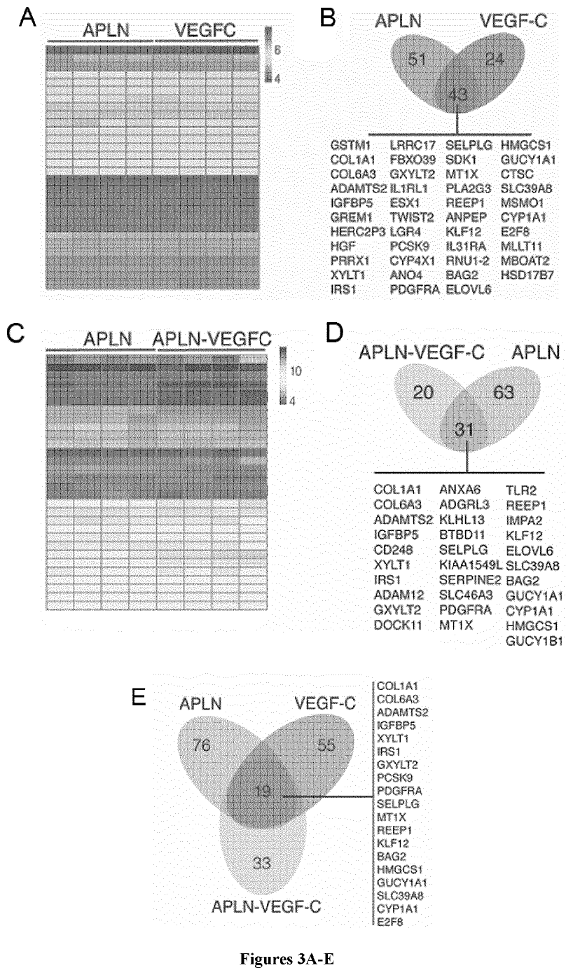

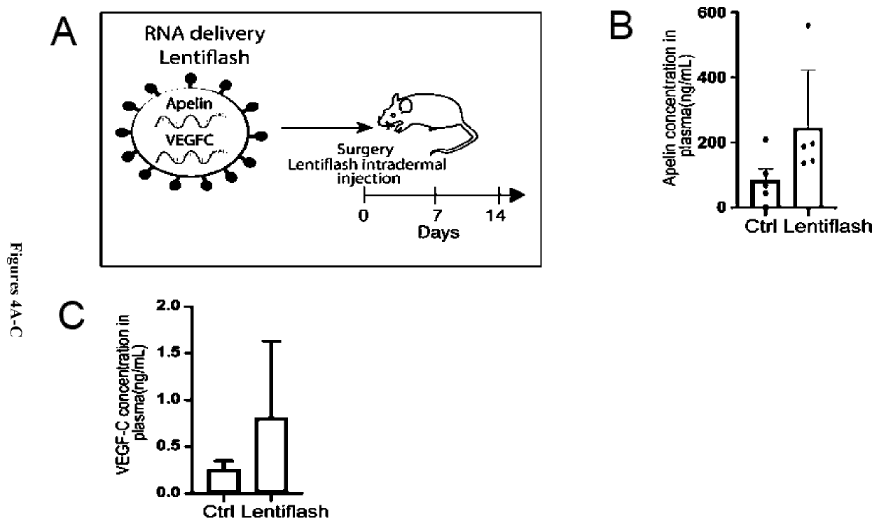

FIG. 3. Apelin and VEGFC exhibit complementary effect on lymphatic endothelial cells
A-E. Comparison of bulk RNA-sequencing in HDLEC treated with apelin-, VEGF-C-, or apelin+VEGF-C-conditioned media. A. Heat map of the top 30 significantly genes up regulated by apelin and VEGF-C. B. Schematic representation of the number of genes up regulated (43) by both apelin and VEGF-C. C. Heat map of the top 30 significantly genes up regulated by apelin and Apelin+VEGF-C treatment. D. Schematic representation of the number of genes up regulated (31) by both apelin and apelin+VEGF-C. E. Schematic representation of the number of genes up regulated (19) by both apelin, VEGF-C and apelin+VEGF-C.

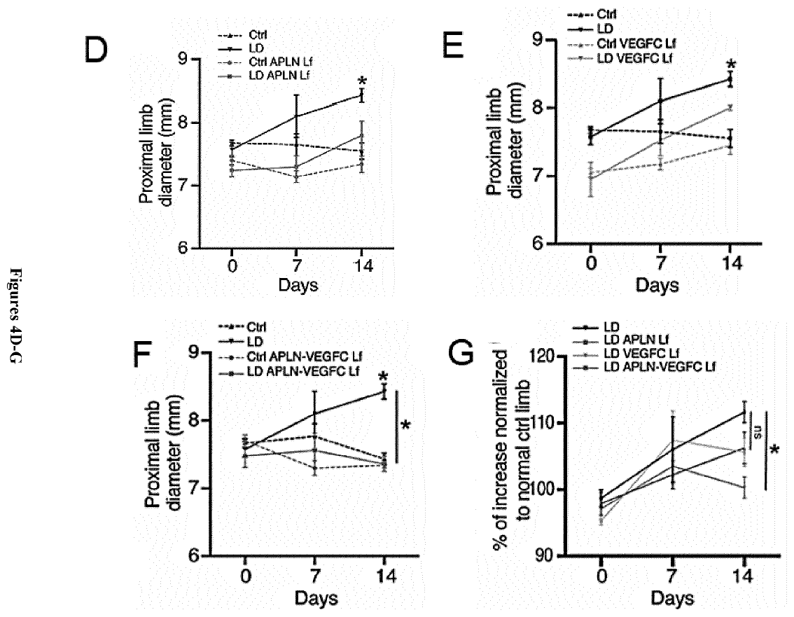

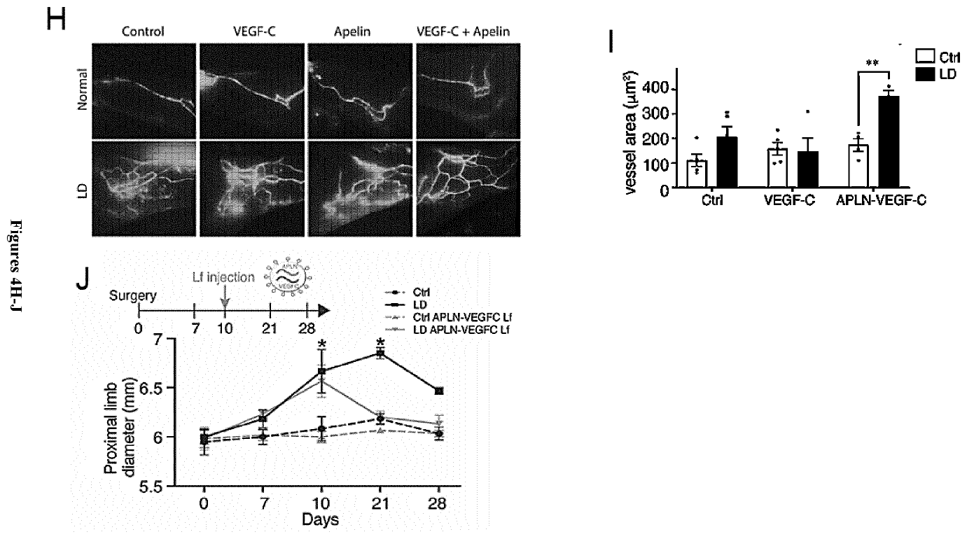

FIG. 4. Apelin-VEGF-C mRNA delivery: a new treatment option for lymphedema
A. Schematic representation of the LentiFlash® vector that encapsulates apelin and VEGF-C mRNA molecules for in vivo delivery. B. EIA dosage of circulating apelin in plasma of control (n=5) or apelin treated mice (n=5). C. EIA dosage of circulating VEGF-C in plasma of control (n=5) or apelin treated mice (n=5). D. Quantification of proximal limb swelling at 7 and 14 days after surgery on control limb, lymphedema limb or lymphedema treated with VEGF-C LentiFlash® vector (n=10) (*p<0.05). E. Quantification of proximal limb swelling at 7 and 14 days after surgery on control limb, lymphedema limb or lymphedema treated with VEGF-C LentiFlash® vector (n=10) (*p<0.05). F. Quantification of proximal leg swelling at 7 and 14 days after surgery on control limb, lymphedema limb or lymphedema treated with apelin-VEGF-C LentiFlash® vector (n=10). G. Quantification of proximal limb swelling 7 and 14 days after surgery on lymphedema limb treated with APLN, VEGFC, or APLN+VEGFC LentiFlash® vector (n=10)(*p<0.05). H. Representatives images of lymphangiography from mice treated with VEGF-C-, apelin-, or apelin-VEGF-C-LentiFlash® vectors. I. Quantification of lymphatic dilatation in apelin-VEGF-C-treated mice (**p<0.01). J. Quantification of proximal limb swelling in mice treated with APLN-VEGF-C LentiFlash® vector after lymphedema development (10 days post-surgery) (n=8) (*p<0.05).

### EXAMPLE

### Methods

### Human Tissue Specimens

Samples were obtained from archival paraffin blocks of 16 lipodermectomy specimens, obtained from patients with secondary lymphedema, treated at Toulouse University Hospital, France, between 2015 and 2016. Patients with a history of 1 to 12 years lymphedema presented stage 2 lymphedema according to the classification of the International Society of Lymphology (ISL). Eligible patients had a history of unilateral non-metastatic breast cancer without recurrence for more than 5 years. The main clinical parameters used to diagnose a significant lymphedema included a clinical upper limb edema with a volume difference between the upper limb affected and the other upper limb of 10% or 200 milliliters (mL). Samples were selected as coded specimens under a protocol approved by the INSERM Institutional Review Board (DC-2008-452), the Research State Department (Ministère de la recherche, ARS, CPP2, authorization AC-2008-452) and the Ethic Committee. When available, some control arm tissue samples removed for esthetical purpose in the same patients, were studied.

### Lymphofluoroscopy

Near-infrared fluorescence lymphatic imaging is used to visualize the initial and conducting lymphatics. 100 μg of indocyanine green (Pulsion®) was diluted in a volume of 0.5 mL of pure water before intradermal injection into the first interdigital space. Fluorescence imaging of the lymphatic flow was observed by fixing the camera (Photo Dynamic Eye, Hamamatsu®) 15 cm above the investigation field. Indocyanine green lymphography findings are classifiable into two patterns: normal linear pattern and abnormal dermal backflow pattern.

### Lymphoscintigraphy

Lymphoscintigraphy was used to diagnose the severity of lymphedema. It is a low radiation examination, forbidden during pregnancy and breastfeeding. Bilateral hypodermal injections were administered between the first and second fingers. The large size of 99m-technetium radiolabeled nanocolloidal albumin were selectively entrapped by lymphatic capillaries and then drained by the 17 lymphatic system. It enabled comparative, functional and bilateral evaluation of the two upper limb including axillary lymph-node uptake, lymphostasis, dermal back flow, and rerouting into the deep lymphatic system into the epitrochlear lymph node.

### Mouse Model of Lymphedema

Mouse procedures were performed in accordance with EU and national regulation. C57B1/6 mouse were provided by Envigo. All experiments have been approved by the local branch Inserm Rangueil-Purpan of the Midi-Pyrénées ethics committee. Secondary lymphedema was established as previously described (Morfoisse et al. 2018). Briefly, lymphedema was established in the left upper limb of 6 weeks-old C57B1/6 female mice. A partial mastectomy of the second mammary gland is performed in association with axillary and brachial lymphadenectomy. Limb size was measured over time in the axillary region using caliper. Mice sustained edema for a period of 2 weeks. The day of the surgery, PBS or apelin lentiviral vector were injected in intradermic in the lymphedema limb (3 injections of 4 μL). For LentiFlash® vector, vehicle, APELIN, VEGFC and APELIN-VEGFC LentiFlash® vectors were injected intradermally into the lymphedema limb (200 ng of p24 splited into 3 injections of 2 μL each). For L-NAME (N^{G}-nitro-L-arginine methyl ester) (Sigma) treatment, L-NAME was resuspended in water (1 mg/ml) and mice were allowed to drink freely for 7 days.

### Lymphangiography

Two weeks after surgery, mice were anesthetized with an intraperitoneal injection of ketamine (100 mg/kg) (Zoletil 100, Virbac) and xylazine (10 mg/kg) (Rompun 2%, Bayer). FITC-Dextran (70 000 kDa, 2 mg/mL, Sigma) was injected into the footpad of lymphedema and control limb. The fluorescent molecule was taken up by the lymphatics and excluded from the blood vessels. After 5 min, the skin was analyzed under the modular stereo microscope discovery. V12 stereo (Zeiss).

### Histology

Skin of lymphedema and control limb were collected 2 weeks after surgery and fixed in 10% neutral buffered formalin O/N at 4° C. Tissue are then embedded in paraffin and sectioned on a microtome. 5 μm sections were cut and placed on Superfrost Plus slides. Tissue were deparaffinized, rehydrated and antigen unmasking was realized with pH 9 Tris solution (H-3301, Vector Laboratories) 5 min three times in a microwave. After cooling, slides were washed in PBS and then blocked with 5% BSA solution at room temperature in humid chamber. Sections were incubated with primary antibodies O/N at 4° C. (Goat anti-murine Lyvel, R&D AF2125; rabbit anti-CD31, abcam Ab28364) and washed three times in PBS. Sections were incubated with the corresponding secondary antibodies conjugated to Alexa-488 or -594 for 1h at room temperature au 1/400 dilution. DNA was stained with DAPI. Slides were mounted with Dako Fluorescence Mounting Medium (S3023). Images were acquired a fluorescent inverted microscope (Leica, DMi8). Images were analysed with Fiji software.

### Evaluation of Fibrosis

Dermis fibrosis was evaluated with Masson's trichrome coloration (MST-100T, Cliniscience). Skin sections were deparraffinized and tissue were stained according the manufacturer's recommendations. Images were acquired on a nanozoomer slide scanner. Dermis size quantification was performed using at least 10 measurements of the length between epidermis and hypodermis per field.

SHG Imaging Skin of lymphedema and control tissues from mice limb were embedded in paraffin and sectioned on a microtome. 30 μm sections were cut and placed on Superfrost Plus slides. Tissue were deparaffinized as described in the “Histology” part and used for SHG analysis. Acquisitions were performed using a Bruker (Billerica, Massachusetts, United States) 2P Plus two-photon microscope. The microscope was equipped with a Coherent (Santa Clara, California, United States) Chameleon Discovery laser, and an Olympus (Shinjuku, Tokyo, Japan) 20×NA:1 objective. We utilized a laser wavelength of 900 nm and collected the Second Harmonic Generation (SHG) emission at 450 nm. Z stacks were acquired with a step size of 1 μm. Collagen fibers quantification was performed using at least 5 measurements per skin section. This analysis was performed on at least 6 different mice per condition.

### Collecting Vessel Contraction Measures

Vessel contraction measurement of afferent collecting lymphatic vessels to the popliteal lymph node (PLN) were performed as described previously (Liao S. et al.). Briefly mice were anesthetized with an intraperitoneal injection of ketamine (100 mg/Kg) and xylazine (10 mg/kg). 6 μL of FITC-Dextran was injected into the footpad of the lower right limb. The skin is carefully removed to expose afferent collecting lymphatic vessels to the PLN. Mouse is then placed into a petri dish and onto the stage of an inverted microscope (Leica, DMi8). 4 videos of 90 sec were acquired per mice and the number of contraction and dilatation (difference between maximum and minimum diameter) was analysed using Fiji software. To assess the effect of Apelin, lentiviral vector was injected into the derm in the lower right limb 7 days before the experiment. For L-NAME study, mice were allowed to drink freely for 7 days.

### LentiFlash® Construction, Production, Purification, and Quantitation by p24 ELISA Assay

Four plasmids were used to produce recombinant LentiFlash® particles in HEK293T cells: (i) the pLVGagPol plasmid coding the viral gag and pol genes modified to harbor the PP7-Coat Protein (PCP) within the gag gene and referred to as pLF-GagPol ΔZF2_PCP (Mianne et al, 2022); (ii) the pVSVG plasmid coding the VSV-G glycoprotein; and (iii) the two plasmids coding each one RNA cargo, flanked by the PP7 bacteriophage aptamers to enable RNA mobilization into lentiviral particles through the interaction with the PP7 coat protein cloned in the Gag sequence. All newly generated constructs were verified by restriction enzyme digestion and sequencing. LentiFlash® particles were produced in a 10-layer CellSTACK chamber (6360cm2, Corning) after transfection of in HEK293T cells with the four plasmids using the standard calcium phosphate procedure. Twenty-four hrs post-transfection, the supernatant was discarded and replaced by fresh medium and cells were incubated at 37° C. in a humidified atmosphere of 5% CO2 in air. After medium change, supernatant was collected, clarified by centrifugation at 3000 g for 5 min, and microfiltered through 0.45-μm pore size sterile filter units (Stericup, Millipore). Supernatant was harvested several times, and finally all samples were pooled (crude harvest). The crude harvest was concentrated and purified by ultrafiltration and diafiltration. For quantification, the p24 core antigen was detected directly in the viral supernatant with a HIV-1 p24 ELISA kit (Perkin Elmer), as specified by the supplier. The viral titer (expressed in physical particles per mL) was calculated from the p24 amount, knowing that 1 pg of p24 corresponds to 10E+4 physical particles.

### Enzyme Immuno Assay (EIA)

Concentration of Apelin in the medium and in mouse plasma was determined using Apelin EIA Kit (RAB0018, Sigma-aldrich) using the manufacturer's recommendations. VEGFC ELISA kit was from R&D systems.

### Cell Culture and Treatments

Human dermal lymphatic endothelial cells (HDLEC) (single donor, juvenile foreskin, Promocell, C-12216, >95% of the cells are CD31 positive and podoplanin positive) were cultured in Endothelial Cell Media MV2 (EGM-MV2, Promocell, C-22121). NIH3T3 were cultured in Dulbecco's modified Eagle's medium (DMEM, Sigma, D6429) supplemented with 10% fetal bovine serum (FBS, Gibco, 10270-06) and 1% penicillin-streptomycin. Endothelial cells were used at passage 3-6 and human fibroblasts were used at passage 4-7. Cells were cultured at 37° C. in a 5% CO₂ incubator. Culture medium was changed 3 times a week, and the cells were passaged ⅓. For collecting conditioned media, NIH3T3 were grown in 10 cm dishes, after reaching confluence, medium was removed and the cells were washed once with PBS. NIH3T3 were cultured overnight in 5 mL reduced serum medium optiMEM (Gibco), the medium was then collected and used for experiments. HDLEC were treated with 50% conditioned media/50% MV2-05% FBS.

RNA Extraction and Reverse Transcriptase and qPCR

Total RNA was prepared using RNeasy kit (Qiagen 74106) according to the supplier's instruction. 1 μg of RNA was retro-transcribed using high-Capacity cDNA Reverse Transcription Kit (Thermo Fisher Scientific, 4368813) containing Multi-Scribe Reverse Transcriptase according to the supplier's instruction. Quantitative real-time PCR was performed using OneGreen FAST qPCR premix (Ozyme, OZYA008) on a StepOne Real-time PCR System (Thermofisher Scientific). All samples were analysed in duplicates. Data was normalized relative to HPRT mRNA levels.

### Immunoblotting

Cells were scrapped and lysed in RIPA buffer (RIPA 2X, Biotech RB4476) supplemented with phosphatase inhibitor (PhosSTOP Easypack, Roche0490687001) and protease inhibitors (protease inhibitor cocktail, Sigma Aldrich). Lysates were centrifuged at 13500 g for 10 minutes at 4° C. Supernatants were then collected and mix with Laemmli buffer containing dithiothreitol (DTT 1 mM). Protein were resolved on 4-15% SDS-PAGE gels and transferred to nitrocellulose membrane (Trans-Blot Turbo RTA transfer kit, #1704271, Biorad). Membranes were blocked for 1h at room temperature in 5% BSA-TBS-T (TBS-0.1% Tween 20) and probed with primary antibody overnight at 4° C. Antibody used are the following: Phospho AKT: AKT-p ser473 (CS #4060S), AKT: santa cruz H136 (S8312), Phospho ERK: ERK1/2-p (MAPKp42/44) (Thr202/Thr204) (cell signalling #9106), ERK ERK1/2-(MAPKp42/44) (Thr202/Thr204) (cell signalling #9102), Phospho eNOS (cell signalling #9571S), eNOS (cell signalling #5880S), E2F8 (Abcam, AB109596), VEGFR3 (RID system AF349), Phospho-VEGFR3 (Affinity AF3676), CCBE1 (Sigma SAB1402017).

After three washes in TBS-T, membranes were probed with HRP-conjugated secondary antibodies at 1/10000 dilution. Signals were visualized with chemiluminescence detection reagent (Sigma) on a Chemidoc (Biorad) digital acquisition system.

### Bulk RNA-Sequencing

### RNA Sequencing on Primary Human Lymphatic Endothelial Cells

Total RNA from HDLEC transduced or not with Apelin Lentivector were prepared using the RNeasy mini kit (Qiagen 74106). Total RNA were then subjected to ribosomal-RNA depleted RNA sequencing (RNA-Seq) protocol performed by Genewiz company using Illumina HiSeq, PE 2×150 configuration. Sequence reads were trimmed to remove possible adapter sequences and nucleotides with poor quality using Trimmomatic v.0.36. The trimmed reads were mapped to the Homo sapiens GRCh38 reference genome available on ENSEMBL using the STAR aligner v.2.5.2b. The STAR aligner is a splice aligner that detects splice junctions and incorporates them to help align the entire read sequences. BAM files were generated as a result of this step. Unique gene hit counts were calculated by using feature Counts from the Subread package v.1.5.2. The hit counts were summarized and reported using the gene_id feature in annotation file. Only unique reads that fell within exon regions were counted. Distribution of read counts in libraries were examined before and after normalization. The original read counts were normalized to adjust for various factors such as variations of sequencing yield between samples. These normalized read counts were used to accurately determine differentially expressed genes. Data quality assessments were performed to detect any samples that are not representative of their group, and thus, may affect the quality of the analysis. The overall similarity among samples were assessed by the Euclidean distance between samples. This method was used to examine which samples are similar/different to each other and if they fit to the expectation from the experiment design. The shorter the distance, the more closely related the samples are. Samples were then clustered by using the distance. A principal component analysis was also performed to reveal the similarity between samples based on the distance matrix.

### Differential Gene Expression Analysis

After extraction of gene hit counts, the gene hit counts table was used for downstream differential expression analysis. Using DESeq2, a comparison of gene expression between Apelin transduced LEC against non-transduced LEC was performed. The Wald test was used to generate p-values and log2 fold changes. Genes with log2FC>0.5 or log2FC<−0.5 and an adjusted p-value <0.05 were defined as differentially expressed genes and used for the downstream analysis. The global transcriptional change across the two groups compared was visualized by a volcano plot. Each data point in the volcano plot represents a gene. The log2 fold change of each gene is represented on the x-axis and the log10 of its adjusted p-value is on the y-axis. Genes with an adjusted p-value less than 0.05 and a log2 fold change greater than 0.5 are indicated by red dots. These represent up-regulated genes. Genes with an adjusted p-value less than 0.05 and a log2 fold change less than 0.5 are indicated by blue dots. These represent down-regulated genes.

### Gene Ontology (GO) Analysis

A gene ontology analysis was performed separately on the statistically significant sets of upregulated and downregulated genes, using PANTHER software (version 16.0, http://pantherdb.org/). The Homo Sapiens reference list was used to cluster the set of significantly differentially expressed genes based on their biological processes or pathways and the overrepresentation of gene ontology terms was tested using Fisher exact test. All the GO terms with a False discovery rate (FDR) lower than 0.05 were considered significant and are listed in Supplementary Data.

### Chromatin Immunoprecipitation (ChIP)

Human dermal lymphatic endothelial cells (HDLEC) non transduced or transduced with Apelin Lentivector, were crosslinked for 15 min using 1% formaldehyde directly in the culture medium. 0.125 M of Glycine were then added for 5 min. After two washes with cold PBS, cells were scraped and frozen at −80° C. Cells were lysed to ChIP-IT Express Magnetic Chromatin

Immunoprecipitation kit (Active Motif 53008). Optimal sonication conditions were determined previously in order to obtain DNA fragments of about 500 bp. Cells were sonicated in 350 μl final volume of Shearing buffer specific to the kit, using Diagenode Bioruptor Sonicator (7 cycles, 30 sec ON, 30 sec OFF in a water bath). DNA concentration was determined using a Nanodrop and 25 μg of chromatin were used by reaction. Experiments were then performed according to the manufacturer protocol. 4 μg of E2F8 antibody (Abcam, AB 109596) were used by ChIP reaction. 10 μl of each sample were kept as Input. Reactions were incubated over night at 4° C. A mock sample without antibody was processed similarly. Prior to qPCR, DNA was purified using the Active Motif Chromatin IP DNA purification kit (58002), and eluted in 50 μl of DNase/RNase free water. 2 μl of purified chromatin was used for qPCR. In some experiments, ChIP reactions were supplemented with 10 ng of Drosophila melanogaster chromatin (spike in chromatin, Active motif, 08221011), and 1 μg of an antibody recognizing H2Av, a Drosophila specific histone variant, (spike in antibody, active motif, 61686), as an internal control for ChIP normalization.

### Cell Transduction

HDLEC were seeded in 6-well plates at 100 000 cells/well. After 24 h, cells were transduced with 1 mL of Apelin lentivector diluted in 1 mL of OptiMEM Media in the presence of proteamine sulfate at a final concentration of 5 μg/mL. The Control cells (NT) non transduced with the Apelin Lentivector were processed at the same time and following the same protocol; in this case 2 mL of OptiMEM and 5 μg/mL of proteamine sulfate were added to the cells. The media was replaced after 24 h. Cells were grown to reach confluence, and then passed and amplified for further experiments. Apelin transduction was verified by RT-qPCR.

### Statistical Analysis

All results presented in this study are representative of at least three independent experiments. In all figures, ‘n’ represents the number of biological replicates. Data are shown as the mean±standard error of the mean (s.e.m.). Statistical significance was determined by two-tailed Student's t test, one-way ANOVA or two-way ANOVA test with Bonferroni post hoc test using Prism ver. 9.0 (GraphPad). Differences were considered statistically significant with a P value <0.05. symbols used are: ns>0.05, *≤0.05, **≤0.01, ***≤0.0001.

### Results

### Secondary Lymphedema Exhibits Increased Lymphatic Capillary Diameter and Poor Collecting Drainage.

Secondary lymphedema occurs months, sometimes years after cancer treatment suggesting that this pathology is not only a side effect of the surgery. Lymphoscintigraphy is the primary imaging modality used to assess lymphatic system dysfunction. It has been considered the criterion standard for decades (Munn & Padera, 2014; Szuba et al, 2003). Lymphoscintigraphy of woman who developed lymphedema after breast cancer reveals a severe decrease in lymphatic collecting vessels detection and axillary lymph node perfusion after radiotracer injection (data not shown). Also, lymphofluoroscopy shows hypervascularized dermis with tortuous lymphatic capillaries (data not shown) associated with a strong desmoplastic reaction and dermal backflow (data not shown). This was confirmed using histological analysis showing an increase of lymphatic vessel density in the skin (FIG. 1A, 1B and data not shown) with hyperplastic overloaded lymphatics (data not shown). Surprisingly, no major difference in genes involved in lymphangiogenic factor maturation was observed except for CCBE1 that was significantly downregulated in lymphedema (FIG. 1C). As lymphedema is characterized by a strong accumulation of fibrotic Adipose Tissue (AT) in the limb, we also evaluated adipokines expression in the lymphedematous AT compared to the normal arm (FIG. 1D). We found a significant decrease in apelin expression in lymphedema whereas no difference was observed for adiponectin expression, leptin expression or other adipokines (FIG. 1D).

### Impaired Lymphatic Healing in APELIN Knocked Out Mice.

APELIN was previously described by our group to improve lymphatic vessel normalization in heart after cardiac ischemia (Tatin et al. 2017). To investigate the role of APELIN in secondary lymphedema, we used a lymphedema mouse model previously developed in the laboratory (Morfoisse et al. 2018). We performed second mammary gland mastectomy associated with axillary and brachial lymphadenectomy on the upper left limb in APELIN-KO mice (data not shown). Using this model, reproducible lymphedema develops after 2 weeks to progressively return to normal after 4 to 8 weeks. In APELIN-KO mice, lymphedema was significantly maintained after 4 weeks showing the failure to restore the lymphatic function (data not shown). Lymphatic capillaries were next investigated using lymphangiography after footpad injection of FITC-Dextran. We observed a strong dermal backflow in APELIN-KO mice 4 weeks after surgery (data not shown). Histological analysis of the lymphatic showed no difference in lymphatic basal density between WT and APELIN-KO mice (data not shown). In contrast, after lymphedema surgery, the skin lymphangiogenesis was significantly reduced in APELIN-KO mice compared to WT mice (data not shown). This was associated with an increase in skin fibrosis in both WT and APELIN-KO mice as shown using Masson's trichrome staining (data not shown). As lymphedema results in an accumulation of collagen fibers, one of the hallmarks of fibrosis development, we performed skin analysis by second harmonic generation (SHG) imaging (data not shown). Interestingly the accumulation of collagen fibers increased in lymphedema in APELIN-KO mice compared to WT mice (data not shown).

### Apelin Possesses Regenerative Function on Lymphatic Vessels in Secondary Lymphedema.

To evaluate the effect of APELIN on lymphatic healing, mice received an intradermic injection of APELIN-expressing lentivector (LV-Apelin) in the lymphedematous limb. Remarkably lymphedema was significantly reduced in Apelin treated mice (FIG. 2A). Level of circulating Apelin was verified by ELISA dosage on mouse plasma showing and increase in plasmatic Apelin concentration in LV-APELIN treated mice (FIG. 2B). Lymphatic collecting drainage was next investigated using lymphangiography (FIG. 2C). Consistently, we observe that secondary lymphedema induces a pathological remodelling of lymphatic vessels with disorganized and abnormal vessel morphology and increased number of branch point regarding the control limb. Lymphatic leakage (dermal backflow) was also observed revealing a dysfunction in superficial capillary network due to the lack of deeper collecting pumping (FIG. 3C). On the contrary, APELIN-treated mice displayed an improvement in the lymphatic shape with normalized morphology and decreased number of vessels branching. Importantly, we did not observe dermal backflow in APELIN-treated mice suggesting an amelioration of lymphatic function (FIG. 3C). Using Masson's trichrome coloration, we observed an increase of the dermis thickness in lymphedema limb consistent with the development of fibrosis (FIG. 2D). In APELIN-treated mice, we did not observe any thickening of the dermis (FIG. 2D and 2E) reflecting an improvement of lymphedema pathology. Interestingly, we observed an increase in circulating VEGF-C, the major lymphangiogenic factor, after LV-APELIN treatment showing that APELIN may in part regulate VEGF-C protein synthesis (FIG. 2F). Positive control was performed using VEGF-C-expressing lentivector (LV-VEGFC)(FIG. 2F). The effect of APELIN was also evaluated on collagen deposition using SHG (data not shown). We found a significant reduction of fibrosis in APELIN-treated mice compared to control (FIG. 2G). The number of blood vessel was assessed using CD31 immunostaining. As expected, we did not find changes in the number of CD31-positive vessel in this model of lymphedema (Morfoisse 2017) (data not shown). However, as previously described in the literature (Wysocka Marta B et al. 2018) treatment with Apelin lentivector led to an increase of angiogenesis and blood vessel permeability (data not shown). In parallel, lymphangiogenesis was evaluated using Lyvel immunostaining on skin sections (FIG. 2H, 2I, 2J). In line with lymphangiography results, an increased number of Lyvel positive vessels was observed in lymphedema limb in comparison to control limb without significant difference when comparing control to LV-Apelin treated mice (FIG. 2I). In contrast, we found that APELIN promotes significant dilatation of lymphatic vessel (FIG. 2J). Overall, our results indicate that Apelin has beneficial role on secondary lymphedema by acting on lymphatic vessel plasticity and dilatation.

### APELIN Controls LEC Gene Expression.

In order to investigate new molecular mechanisms and signalling pathway regulated by Apelin in LEC, we performed a global transcriptomic analysis on LEC stimulated 24 with conditioned media containing APELIN or conditioned media obtained from control NIH3T3 (data not shown). RNA-sequencing samples quality and similarity assessment were verified (data not shown). Differential DESeq analysis revealed that 217 genes were deregulated (p.adj<0.05 and Log2 fold change<−0.5 or >0.5) with 94 genes up regulated and 123 genes down regulated (data not shown). Top 30 down or up regulated are display on heat maps (data not shown) and complete list are given in data not shown. Gene ontology analysis of down regulated genes revealed that no biological process was significantly affected in Apelin treated HDLEC. In contrast, GO analysis for biological process revealed that upregulated genes were enriched (FDR<0.05) for terms related to extracellular matrix (ECM) remodelling and signalisation (data not shown) including COL1A, FBN, ADATS2, and CCBE1 (data not shown). However, most of these gene induction was not validated by RT-qPCR on HDLEC except for collagen- and calcium-binding EGF domains 1 (CCBE1) whose induction was strongly confirmed (data not shown). CCBE1 protein is required for the activation of VEGF-C along with the ADAMTS3 (A Disintegrin And Metalloproteinase with Thrombospondin Motifs-3) protease by enhancing the cleavage activity of ADAMTS3 and by facilitating the maturation of VEGF-C into its bioactive form. To investigate the effect of CCBE1 on VEGFC receptor activation, we performed the knock-down of CCBE1 in LEC using siRNA (data not shown). Then, cells were stimulated by APELIN and western blot analysis of P-VEGFR3 was performed (data not shown). We found that the knock-down of CCBE1 in LEC decreases the amount of VEGFR-3 protein. This was associated with a slight, but significant decrease of VEGFR-3 phosphorylation in the presence of APELIN (data not shown). Interestingly, in APELIN also stimulated the expression of E2F8, the CCBE1 transcription factor (data not shown). We then postulated that APELIN could participate to VEGF-C maturation by increasing E2F8 DNA binding on CCBE1 promoter (data not shown). To answer to this question, we performed chromatin immunoprecipitation (ChIP) experiment using E2F8 immunoprecipitation on APELIN-overexpressing HDLEC (data not shown). We found that apelin significantly increased E2F8 binding to CCBE1 promoter (data not shown). Interestingly, apelin also induced E2F8 binding to E2F1 transcription factor promoter suggesting a role in other biological functions (data not shown) (Wells, Graveel et al. 2002). However, no binding on FLT4 was found (data not shown). Altogether, these data indicated that APELIN regulates HDLEC gene expression.

### APELIN Stimulates LEC Function Through Akt/eNOS Signalling.

Next we investigated which molecular pathway is involved in response to apelin in vitro. Apelin is known to activate Erk and Akt signalling in vitro in Human Dermal Lymphatic Endothelial cells (HDLEC) (Kim, Kang et al. 2014) (Berta, Hoda et al. 2014). In line with the vasodilation phenotype (data not shown), we postulated that APELIN beneficial effect on lymphedema is in part mediated by AKT/eNOS pathway. To this end, we stimulated HDLEC with conditioned media obtained from LV-Apelin transduced NIH3T3 previously depleted for VEGF-C. Apelin synthesis was validated by RT-qPCR on NIH3T3 (data not shown) and by ELISA dosage (data not shown). Stimulation of HDLEC by conditioned media was confirm by evaluating AKT et ERK pathway during 24 h time course and media containing VEGF-C was used as positive control (data not shown). In that context, LEC responded to VEGF-C after 30 min as we observed a strong activation of AKT and ERK (data not shown) whereas APELIN stimulated Akt phosphorylation after 1 hour without major effect on ERK (data not shown). Importantly, eNOS phosphorylation was observed in HDLEC in response APELIN and VEGF-C (data not shown). In line with Akt activation in HDLEC, we found an activation of cell migration (data not shown), whereas no effect was observed on cell junction or cytoskeleton remodeling (data not shown). Importantly, eNOS phosphorylation was observed in HDLEC in response to APELIN and/or VEGFC, suggesting that both APELIN and VEGF-C stimulate lymphatic dilatation through eNOS pathway (data not shown).

Apelin Stimulates Lymphatic Pumping Through eNOS Activation.

We next explored whether apelin could control vessel dilatation, in particular its role on collecting lymphatic vessels. apelin was described to activate eNOS phosphorylation in several cellular contexts to promote blood vessel dilatation (Dray, Knauf et al. 2008) (Wysocka, Pictraszek-Gremplewicz et al. 2018). We then investigated whether APELIN was able to stimulate lymphatic collecting vessels dilatation and thus lymphatic pumping (data not shown). Lymph flow is in part driven in collecting lymphatics by autonomous contractions of smooth muscle cells. To evaluate the effect of apelin on collecting vessel contractions, we used intravital imaging method previously described (Liao, Jones et al. 2014)(data not shown). Number of contraction and the dilatation of vessels was assessed. Interestingly, we found that apelin stimulated lymphatic pumping by increasing the collector dilatation (data not shown) without any effect on contraction frequency (data not shown). This effect was completely reversed by the L-NAME, the nitric oxide synthase (NOS) inhibitor (data not shown). Then, to test the role of eNOS activation in response to apelin in vivo in lymphedema context, LV-apelin treated mice were submitted to L-NAME treatment (data not shown). Limb diameter was measured to assess the edema (data not shown). Interestingly, L-NAME reversed the beneficial effect of apelin on lymphedema confirming the lymphatic pumping as a major etiology of the pathology. We also observed a significant increase of edema two weeks after surgery in the presence of apelin+L-NAME (data not shown). Lymphangiography revealed that L-NAME treatment also reverse apelin effect on lymphatic vascular network. Indeed, in APELIN+L-NAME treated mice, we observe pathological remodeling of lymphatic vessel with dermal backflow and abnormal lymphatic branching (data not shown). Capillaries were also quantified using Lyve1 immunodetection on skin sections (data not shown). An increase of lymphangiogenesis was observed systematically in lymphedema limb, however we did not observe any differences between conditions (data not shown). Nevertheless, regarding vessel area, apelin-treated mice displayed an increased dilatation that was inhibited by L-NAME treatment restoring a similar phenotype compared to control group (data not shown). We also investigated fibrosis and surprisingly L-NAME has no effect on fibrosis (data not shown). Taken together our result show that Apelin prevent lymphedema by promoting collecting vessels pumping and pathological remodelling of lymphatic vessel. This phenotype seems to be mediated in part by activating eNOS in lymphatic endothelial cells.

### APELIN and VEGFC Exhibit a Synergistic Effect on the Regulation of Gene Expression Related to Collecting Vessels Maintenance.

Most of the studies aiming at regenerating the lymphatic system have focused on VEGF-C molecule. Nevertheless, VEGFC alone has appeared ineffective to improve lymphatic function in mice model of vascular injury suggesting that it has to be combined with other molecules to fully restore the lymphatic function. Here we found that APELIN controlled lymphedema fibrosis, lymphatic function and contractility of collecting vessels. Our original approach aims at combining VEGF-C with apelin to obtain a synergistic effect for the treatment of secondary lymphedema by targeting the entire lymphatic network from capillaries to collectors. When comparing gene expression profile of APELIN-, VEGF-C or APELIN+VEGF-C stimulated HDLEC, we observed similar induction of top 30 genes mostly related to extracellular matrix remodeling (data not shown). The majority of the genes induced by VEGF-C are also induced by APELIN (FIG. 3A, 3B). Half of the genes induced by the combination of APELIN+VEGF-C are induced by APELIN (FIG. 3C, 3D). When comparing induction of genes shared by the two molecules, the cooperative effect of APELIN and VEGF-C remained focused on genes related to the microenvironmental maintenance of the lymphatic system (collagen 1A1 and 6A) and to the maturation of VEGF-C (ADAMTS2, E2F8)(FIG. 3E). Also, 33 genes are specifically upregulated by the combination of APELIN and VEGF-C (FIG. 3E). When comparing non treated-, VEGFC alone or APELIN alone to APELIN+VEGFC combination, we found an increase of genes necessary for collecting vessel function including connexin 37 and 47 (GJA4, GJC2) and claudin 5 (CDN5), whereas angiogenic genes were down-regulated (VEGFA, FLT1, KDR, KI67)(data not shown). The expression of genes related to VEGFC maturation (ADAMTS2, CCBE1) was improved in VEGFC-, APELIN-, and APELINVEGFC groups compared to WT (data not shown).

### APELIN-VEGFC RNA Delivery: a New Therapeutic Option for Secondary Lymphedema.

In western countries, secondary lymphedema develops after cancer treatment, which makes ethical concern for the delivery of angiogenic molecules to cancer survival patients. For security reasons, we decided to use the next generation of vector called LentiFlash® (Lf) which allows mRNA transient delivery from non-integrative particles. Based on LentiFlash® capacity for delivering several heterologous mRNA molecules, we generated a LentiFlash® vector containing two different mRNAs coding for VEGF-C or APELIN, respectively, to be injected in the mouse model of lymphedema (FIG. 4A). LentiFlash® efficiency is highly dependent on mRNA stability compared to lentivector that induces permanent expression of the transgene without any effect on immune cell populations of platelet numbers (data not shown). We therefore first confirmed the expression of circulation VEGF-C (FIG. 4B) and apelin (FIG. 4C) by ELISA, measurable 48 hrs after injection. As expected, LentiFlash® mRNA delivery was not as efficient as lentivector as we only observed a partial inhibition of limb swelling using VEGF-C or apelin single mRNA delivery (FIG. 4D, 4E). This could be expected, due to the restricted time of molecule expression. However, the VEGF-C/apelin double mRNA LentiFlash® completely abolished limb swelling (FIG. 4F, 4G), reduced dermal backflow (FIG. 4H) and restored the lymphatic perfusion in the lymphedematous limb (FIG. 4I). This was associated with an increase in lymphatic vessel diameter (FIG. 4I). Finally, to investigate whether Apelin-VEGFC mRNA could be a curative treatment for lymphedema, we injected mice which developed lymphedema (10 days after surgery) (FIG. 4J). In that context, LentiFlash® vector reversed lymphedema swelling to go back to normal after 11 days. These data showed the synergistic effect of Apelin and VEGFC and demonstrated that this combination generates a significant therapeutic benefit despite the transient expression of the two transgenes, providing a perspective of lymphedema treatment using nonintegrative RNA delivery vectors for patients who develop lymphedema after cancer treatment. Thus, apelin represents highly efficient molecule to combine to VEGF-C in the treatment of lymphedema using “safe” RNA delivery vectors for patients who develop lymphedema after cancer treatment.

### Discussion

Despite large advances in the past decades for the understanding of the molecular mechanisms that drive the lymphatic function, lymphedema, the most predominant pathology associated with lymphatic dysfunction remains an unmet medical need (Mercier, Pastor et al. 2019). It is a painful chronic condition that affects millions of people worldwide. Many factors contribute to the etiology of the disease. Primary lymphedema, an inherited disease, is induced by genetic mutation, whereas secondary lymphedema occurs after cancer treatment or filarial infection (Mortimer and Rockson 2014, Rockson 2018). However, they all lead to comparable clinical signs: an accumulation of fluid and fat in the limb associated with fibrosis and hypervascularized dermis characterized by tortuous and leaky capillaries and hypoperfusion of deeper collecting vessels. Lymphoscintigraphies show a severe reduction of lymph node perfusion demonstrating that the lymphatic collecting vessels are still present, but can not collect and drive the lymph properly. These observations support the therapeutic strategy aiming at combining molecules to 1/normalize the capillary territories and 2/regenerate the lymphatic pumping in deeper adipose depots. Whereas it is now well established that VEGF-C, the major lymphangiogenic growth factor, is the best candidate the restore the lymphatic capillary network (Hartiala, Suominen et al. 2020), its role on collecting remains less effective. VEGF-C binds to its tyrosine kinase receptor VEGFR-3 to promote biological activities (Alitalo, Tammela et al. 2005). Collecting vessels develop in an integrated adipose environment that is considerably modified during lymphedema. In particular, adipose tissue synthesize many adipokines involved in the blood and lymphatic vessel integrity. It is therefore tempting to speculate that changes in adipokine production may affect the lymphatic collecting function. Among them, apelin has been described to be a key factor for stimulating LEC function (Kim, Kang et al. 2014). Apelin stimulates lymophangiogenesis in cancer and participates to the restoration of pre-collecting lymphatics shape after myocardial infarction (Tatin, Renaud-Gabardos et al. 2017). Apelin is a bioactive peptide that induces signalling after binding to its G protein-coupled receptor APJ located at the surface of LEC. In addition to its effect on the endothelial monolayer, apelin is a robust antifibrotic molecule (Huang, Chen et al. 2016).

By performing gene expression analysis of dermolipectomies from women who developed secondary lymphedema after breast cancer, we identified a significant decrease of apelin expression in lymphedema. The crucial role of apelin in lymphedema was confirmed in apelin KO mice that exhibits an aggravation of lymphedema that can be restored by an apelin-expressing lentivector. In the mouse model of lymphedema, we identified that apelin improves lymphedema condition by acting on two major hallmarks of the pathology: lymphatic function and tissue fibrosis. Importantly, we found that the effect of apelin on the lymphatic collecting pumping was directly controlled by the NOS. NO production participates to the endothelial homeostasis by controlling the modulation of vascular tone as an adaptation of flow (Dimmeler, Fleming et al. 1999). eNOS mediates key aspects in vascular remodeling by translating mechanical stimuli into enhance NO production. The endothelial NOS (eNOS) also regulates lymphatic homeostasis. In a mouse model of fibrosarcoma, eNOS mediates VEGF-C induced lymphangiogenesis and tumor lymphatic metastasis (Lahdenranta, Hagendoorn et al. 2009). Other studies have shown that eNOS affects the lymph flow via the collecting lymphatics, whithout affecting the diameter of capillaries (Hagendoorn, Padera et al. 2004). Also, NO bioavailability in pulmonary lymphatics was found to be impaired in lambs that exhibit chronically increased pulmonary blood and lymph flow (Datar, Gong et al. 2016). Here, we identified that the effect of apelin on the lymphatic collectors pumping is mediated by eNOS. Importantly, apelin was previously described to modulates the aortic vascular tone by increasing the phosphorylation of Akt and eNOS in diabetic mice (Zhong, Yu et al. 2007). Here, we identified that the beneficial effect of apelin on lymphatic collecting vessels was mediated by this pathway suggesting that apelin can be at the origin of NO-mediated lymphatic pumping in many organs. The Akt-phosphorylation was found in a lesser extent than the phosphorylation induced by VEGF-C, however, it seems to be efficient to mediate its biological effects. Interestingly, no effect of apelin was found on the endothelial monolayer integrity, suggesting that the role of apelin is restricted to functional and dynamic effect.

The RNA sequencing on apelin-stimulated LEC revealed that apelin controls the expression of genes involved in extracellular matrix remodeling in line with its effect on tissue fibrosis. Interestingly, apelin also strongly stimulated the expression of CCBE1, a protein involved in the proteolytic activation of VEGF-C by ADAMTS3 (Jha, Rauniyar et al. 2017). This could in part explain the increase of circulating VEGF-C concentration observed after apelin treatment. Importantly, in human, mutations in CCBE1 were found to cause Hennekam syndrome, a congenital disease characterized lymphatic malformations leading to primary lymphedema, lymphangiectasia, and heart defects (Alders, Mendola et al. 2013). Mecanistically, we found that CCBE1 gene expression is controlled by apelin that directly increase the fixation of E2F8, its transcription factor, on the promoter. Altogether, these data reinforce the apelin as a key factor in restoring the lymphatic function in lymphedema. Therefore, we proposed to evaluate the effect of apelin coupled to VEGF-C in a Phase I clinical trial for secondary lymphedema that will be launched in Toulouse hospital. This pilot study called Theralymph will focus on women who develop lymphedema after breast cancer. However, an important ethical issue in treated cancer survivor patients is to reactivate the tumor with pro-lymphangiogenic therapy, even with more than five years without any recurrence. Therefore, the use of permanent integration of the transgene using lentiviral gene therapy rapidly appeared as not an optimal solution for treatment delivery. Another issue is the brief plasma apelin half-life which is less than five minutes (Japp and Newby 2016). This could have been compensated by successive injections in the limb, however it would significantly improve the risk of infections and desmoplastic reaction, which are often seen in lymphedema patients. The adeno-associated virus was also dropped out due to the limitation of transgene size, which makes impossible the simultaneous delivery of multiple therapy. We then decided to use the LentiFlash® vector, a novel class of non-integrative lentivector that allow the delivery of transient multiple mRNA particles (Prel, Caval et al. 2015). LentiFlash® is constructed using a bacteriophage coat protein and its cognate 19-nt stem loop, to replace the natural lentiviral Psi packaging sequence, in order to achieve active mRNA packaging into the lentiviral particles. Single apelin mRNA LentiFlash® exhibits less efficiency in reducing lymphedema compared to integrative lentivector. However, when combined to VEGF-C, double mRNA delivery completely abolished lymphedema and restored the lymphatic flow in the limb showing that mRNA delivery strategy allows enough synthesis of the two proteins to observe a beneficial effect.

In the past two years, we have seen the emergence of a novel class of mRNA vaccine, a highly efficient and low-toxic vectors. We believe that mRNA can treat many diseases including lymphedema, a pathology with currently no treatments, in a different way that traditional medicine. The plasticity of the LentiFlash® allows the transient delivery of 2 mRNA molecules allowing here to stimulate the synergistic effect of APJ, a G protein-coupled receptor with VEGFR-3, a tyrosine kinase receptor. Based on the fact that lymphedema remains a multifactorial pathology with lymphatic endothelial dysfunction, adipose tissue accumulation and fibrosis, we are convinced that multiple therapy will be the solution to cure this harmful condition. Therefore, we proposed to use the apelin-VEGF-C LentiFlash® vector for a Phase I/II gene therapy clinical trial that will be launched in our hospital next year.

### REFERENCES

Throughout this application, various references describe the state of the art to which this invention pertains. The disclosures of these references are hereby incorporated by reference into the present disclosure.
Alders, M., A. Mendola, L. Ades, L. Al Gazali, C. Bellini, B. Dallapiccola, P. Edery, U. Frank, F. Hornshuh, S. A. Huisman, S. Jagadeesh, H. Kayserili, W. T. Keng, D. Lev, C. E. Prada, J. R. Sampson, J. Schmidtke, V. Shashi, Y. van Bever, N. Van der Aa, J. M. Verhagen, J. B. Verheij, M. Vikkula and R. C. Hennekam (2013). “Evaluation of Clinical Manifestations in Patients with Severe Lymphedema with and without CCBE1 Mutations.” Mol Syndromol 4(3): 107-113.
Alitalo, K., T. Tammela and T. V. Petrova (2005). “Lymphangiogenesis in development and human disease.” Nature 438(7070): 946-953.
Aschen, S., J. C. Zampell, S. Elhadad, E. Weitman, M. De Brot Andrade and B. J. Mehrara (2012). “Regulation of adipogenesis by lymphatic fluid stasis: part II. Expression of adipose differentiation genes.” Plast Reconstr Surg 129(4): 838-847.
Baik, J. E., H. J. Park, R. P. Kataru, I. L. Savetsky, C. L. Ly, J. Shin, E. M. Encarnacion, M. R. Cavali, M. G. Klang, E. Riedel, M. Coriddi, J. H. Dayan and B. J. Mehrara (2022). “TGF-beta1 mediates pathologic changes of secondary lymphedema by promoting fibrosis and inflammation.” Clin Transl Med 12(6): e758.
Berta, J., M. A. Hoda, V. Laszlo, A. Rozsas, T. Garay, S. Torok, M. Grusch, W. Berger, S. Paku, F. Renyi-Vamos, B. Masri, J. Tovari, M. Groger, W. Klepetko, B. Hegedus and B. Dome (2014). “Apelin promotes lymphangiogenesis and lymph node metastasis.” Oncotarget 5(12): 4426-4437.
Busch, R., A. Strohbach, M. Pennewitz, F. Lorenz, M. Bahls, M. C. Busch and S. B. Felix (2015). “Regulation of the endothelial apelin/APJ system by hemodynamic fluid flow.” Cell Signal 27(7): 1286-1296.
Castan-Laurell, I., J. Boucher, C. Dray, D. Daviaud, C. Guigne and P. Valet (2005). “Apelin, a novel adipokine over-produced in obesity: friend or foe?” Mol Cell Endocrinol 245(1-2): 7-9.
Dai, L., P. M. Smith, M. Kuksis and A. V. Ferguson (2013). “Apelin acts in the subfornical organ to influence neuronal excitability and cardiovascular function.” J Physiol 591(13): 3421-3432.
Datar, S. A., W. Gong, Y. He, M. Johengen, R. J. Kameny, G. W. Raff, E. Maltepe, P. E. Oishi and J. R. Fineman (2016). “Disrupted NOS signaling in lymphatic endothelial cells exposed to chronically increased pulmonary lymph flow.” Am J Physiol Heart Circ Physiol 311(1): H137-145.
Dimmeler, S., I. Fleming, B. Fisslthaler, C. Hermann, R. Busse and A. M. Zeiher (1999). “Activation of nitric oxide synthase in endothelial cells by Akt-dependent phosphorylation.” Nature 399(6736): 601-605.
Dray, C., C. Knauf, D. Daviaud, A. Waget, J. Boucher, M. Bulcon, P. D. Cani, C. Attane, C. Guigne, C. Carpene, R. Burcelin, I. Castan-Laurell and P. Valet (2008). “Apelin stimulates glucose utilization in normal and obese insulin-resistant mice.” Cell Metab 8(5): 437-445.
Greene, A. K., F. D. Grant and S. A. Slavin (2012). “Lower-extremity lymphedema and elevated body-mass index.” N Engl J Med 366(22): 2136-2137.
Gupta, R., J. Tongers and D. W. Losordo (2009). “Human studies of angiogenic gene therapy.” Circ Res 105(8): 724-736.
Hagendoorn, J., T. P. Padera, S. Kashiwagi, N. Isaka, F. Noda, M. I. Lin, P. L. Huang, W. C. Sessa, D. Fukumura and R. K. Jain (2004). “Endothelial nitric oxide synthase regulates microlymphatic flow via collecting lymphatics.” Circ Res 95(2): 204-209.
Hartiala, P., S. Suominen, E. Suominen, I. Kaartinen, J. Kiiski, T. Viitanen, K. Alitalo and A. M. Saarikko (2020). “Phase 1 Lymfactin( ) Study: Short-term Safety of Combined Adenoviral VEGF-C and Lymph Node Transfer Treatment for Upper Extremity Lymphedema.” J Plast Reconstr Aesthet Surg 73(9): 1612-1621.
Hoffner, M., P. Peterson, S. Mansson and H. Brorson (2018). “Lymphedema Leads to Fat Deposition in Muscle and Decreased Muscle/Water Volume After Liposuction: A Magnetic Resonance Imaging Study.” Lymphat Res Biol 16(2): 174-181.
Hogan, B. M., F. L. Bos, J. Bussmann, M. Witte, N. C. Chi, H. J. Duckers and S. Schulte-Merker (2009). “Cebel is required for embryonic lymphangiogenesis and venous sprouting.” Nat Genet 41(4): 396-398.
Huang, S., L. Chen, L. Lu and L. Li (2016). “The apelin-APJ axis: A novel potential therapeutic target for organ fibrosis.” Clin Chim Acta 456: 81-88.
Japp, A. G. and D. E. Newby (2016). “Unlocking the Therapeutic Potential of Apelin.” Hypertension 68(2): 307-309.
Jha, S. K., K. Rauniyar, T. Karpanen, V. M. Leppanen, P. Brouillard, M. Vikkula, K. Alitalo and M. Jeltsch (2017). “Efficient activation of the lymphangiogenic growth factor VEGF-C requires the C-terminal domain of VEGF-C and the N-terminal domain of CCBE1.” Sci Rep 7(1): 4916.
Kataru, R. P., I. Wiser, J. E. Baik, H. J. Park, S. Rchal, J. Y. Shin and B. J. Mehrara (2019). “Fibrosis and secondary lymphedema: chicken or egg?” Transl Res 209: 68-76.
Kim, J. D., Y. Kang, J. Kim, I. Papangeli, H. Kang, J. Wu, H. Park, E. Nadelmann, S. G. Rockson, H. J. Chun and S. W. Jin (2014). “Essential role of Apelin signaling during lymphatic development in zebrafish.” Arterioscler Thromb Vasc Biol 34(2): 338-345.
Koc, M., M. Wald, Z. Varaliova, B. Ondrujova, T. Cizkova, M. Brychta, J. Kracmerova, L. Beranova, J. Pala, V. Sramkova, M. Siklova, J. Gojda and L. Rossmeislova (2021). “Lymphedema alters lipolytic, lipogenic, immune and angiogenic properties of adipose tissue: a hypothesis-generating study in breast cancer survivors.” Sci Rep 11(1): 8171.
Lahdenranta, J., J. Hagendoorn, T. P. Padera, T. Hoshida, G. Nelson, S. Kashiwagi, R. K. Jain and D. Fukumura (2009). “Endothelial nitric oxide synthase mediates lymphangiogenesis and lymphatic metastasis.” Cancer Res 69(7): 2801-2808.
Lammens, T., J. Li, G. Leone and L. De Veylder (2009). “Atypical E2Fs: new players in the E2F transcription factor family.” Trends Cell Biol 19(3): 111-118.
Liao, S., D. Jones, G. Cheng and T. P. Padera (2014). “Method for the quantitative measurement of collecting lymphatic vessel contraction in mice.” J Biol Methods 1(2).
Logan, N., A. Graham, X. Zhao, R. Fisher, B. Maiti, G. Leone and N. B. La Thangue (2005). “E2F-8: an E2F family member with a similar organization of DNA-binding domains to E2F-7.” Oncogene 24(31): 5000-5004.
Mercier, G., J. Pastor, V. Clement, U. Rodts, C. Moffat and I. Quere (2019). “Out-of-pocket payments, vertical equity and unmet medical needs in France: A national multicenter prospective study on lymphedema.” PLOS One 14(5): e0216386.
Mortimer, P. S. and S. G. Rockson (2014). “New developments in clinical aspects of lymphatic disease.” J Clin Invest 124(3): 915-921.
Pope, G. R., E. M. Roberts, S. J. Lolait and A. M. O'Carroll (2012). “Central and peripheral apelin receptor distribution in the mouse: species differences with rat.” Peptides 33(1): 139-148.
Prel, A., V. Caval, R. Gayon, P. Ravassard, C. Duthoit, E. Payen, L. Maouche-Chretien, A. Creneguy, T. H. Nguyen, N. Martin, E. Piver, R. Sevrain, L. Lamouroux, P. Leboulch, F. Deschaseaux, P. Bouille, L. Sensebe and J. C. Pages (2015). “Highly efficient in vitro and in vivo delivery of functional RNAs using new versatile MS2-chimeric retrovirus-like particles.” Mol Ther Methods Clin Dev 2: 15039.
Rockson, S. G. (2018). “Lymphedema after Breast Cancer Treatment.” N Engl J Med 379(20): 1937-1944.
Sano, M., S. Hirakawa, T. Sasaki, K. Inuzuka, K. Katahashi, T. Kayama, Y. Yamanaka, H. Tsuyuki, Y. Endo, E. Naruse, Y. Yokoyama, K. Sato, K. Yamauchi, H. Takeuchi and N. Unno (2022). “Role of Subcutaneous Adipose Tissues in the Pathophysiology of Secondary Lymphedema.” Lymphat Res Biol.
Sawane, M., K. Kajiya, H. Kidoya, M. Takagi, F. Muramatsu and N. Takakura (2013). “Apelin inhibits diet-induced obesity by enhancing lymphatic and blood vessel integrity.” Diabetes 62(6): 1970-1980.
Tatin, F., E. Renaud-Gabardos, A. C. Godet, F. Hantelys, F. Pujol, F. Morfoisse, D. Calise, F. Viars, P. Valet, B. Masri, A. C. Prats and B. Garmy-Susini (2017). “Apelin modulates pathological remodeling of lymphatic endothelium after myocardial infarction.” JCI Insight 2(12).
Van Balkom, I. D., M. Alders, J. Allanson, C. Bellini, U. Frank, G. De Jong, I. Kolbe, D. Lacombe, S. Rockson, P. Rowe, F. Wijburg and R. C. Hennekam (2002). “Lymphedema-lymphangiectasia-mental retardation (Hennekam) syndrome: a review.” Am J Med Genet 112(4): 412-421.
Weijts, B. G., A. van Impel, S. Schulte-Merker and A. de Bruin (2013). “Atypical E2fs control lymphangiogenesis through transcriptional regulation of Ccbe1 and Flt4.” PLOS One 8(9): e73693.
Wells, J., C. R. Graveel, S. M. Bartley, S. J. Madore and P. J. Farnham (2002). “The identification of E2F1-specific target genes.” Proc Natl Acad Sci U S A 99(6): 3890-3895.
Wysocka, M. B., K. Pietraszek-Gremplewicz and D. Nowak (2018). “The Role of Apelin in Cardiovascular Diseases, Obesity and Cancer.” Front Physiol 9: 557.
Yokoyama, Y., A. Sekiguchi, C. Fujiwara, A. Uchiyama, A. Uehara, S. Ogino, R. Torii, O. Ishikawa and S. I. Motegi (2018). “Inhibitory Regulation of Skin Fibrosis in Systemic Sclerosis by Apelin/APJ Signaling.” Arthritis Rheumatol 70(10): 1661-1672.
Zampell, J. C., S. Aschen, E. S. Weitman, A. Yan, S. Elhadad, M. De Brot Andrade and B. J. Mehrara (2012). “Regulation of adipogenesis by lymphatic fluid stasis: part I. Adipogenesis, fibrosis, and inflammation.” Plast Reconstr Surg 129(4): 825-834.
Zhong, J. C., X. Y. Yu, Y. Huang, L. M. Yung, C. W. Lau and S. G. Lin (2007). “Apelin modulates aortic vascular tone via endothelial nitric oxide synthase phosphorylation pathway in diabetic mice.” Cardiovasc Res 74(3): 388-395.

1. A method of treating lymphedema in a patient in need thereof comprising administering to the patient a therapeutically effective amount of i) an apelin polypeptide or ii) a polynucleotide encoding an apelin polypeptide.

2. The method of claim 1 wherein the patient suffers from a secondary lymphedema.

3. The method of claim 1 wherein the apelin polypeptide or the polynucleotide encoding the apelin polypeptide is suitable for increasing lymphatic vessel plasticity, contractility and/or dilatation.

4. The method of claim 1 wherein the polypeptide comprises an amino acid sequence having at least 90% of identity with the amino acid sequence as set forth in SEQ ID NO:1.

5. The method of claim 1 wherein the polynucleotide encodes an amino acid sequence having at least 90% of identity with the amino acid sequence as set forth in SEQ ID NO:1.

6. The method of claim 1 wherein the polynucleotide is a messenger RNA (mRNA).

7. The method of claim 1 wherein the polynucleotide is inserted in a vector, such a viral vector.

8. The method of claim 4 wherein the viral vector is a AAV vector.

9. The method of claim 4 wherein the viral vector is a retroviral vector.

10. The method of claim 6 wherein the retroviral vector is a lentiviral vector.

11. The method of claim 1 wherein the polypeptide or polynucleotide is encapsulated in a virus-like particle.

12. The method of claim 1 wherein the polypeptide or polynucleotide is administered in combination or association with a VEGF-C polypeptide or ii) a polynucleotide encoding a VEGF-C polypeptide.


SEQUENCE LISTING (2 sequences)

SEQ ID NO: 1 - protein, 77 aa, Homo sapiens
MNLRLCVQALLLLWLSLTAVCGGSLMPLPDGNGLEDGNVRHLVQPRGSRNGPGPWQGGRRKFRRQRPRLSHKGPMPF

SEQ ID NO: 2 - protein, 419 aa, Homo sapiens
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS